(19) United States
(12) Patent Application Publication
(10) Pub. No.: US 2025/0152496 A1
(43) Pub. Date: May 15, 2025

(54) SKIN IMPROVING AGENT CONTAINING FERMENTED EXTRACT AS MAIN RAW MATERIAL

(71) Applicant: AIN HOLDINGS INC., Sapporo (JP)

(72) Inventors: Takumi WATANABE, Sayama (JP); Yong-Jin CHANG, Himeji (JP); Chiori SATO, Tokyo (JP)

(73) Assignee: AIN HOLDINGS INC., Sapporo (JP)

(21) Appl. No.: 18/838,656

(22) PCT Filed: Oct. 25, 2023
(86) PCT No.: PCT/JP2023/038462

§ 371 Date: Aug. 15, 2024

(30) Foreign Application Priority Data
Oct. 25, 2022 (JP) 2022-170889

(51) Int. Cl. A61K 8/9728; A61K 8/99; C12N 1/20; C12R 1/46

(52) U.S. Cl. CPC A61K 8/9728; A61K 8/99; C12N 1/20; A61K 2800/782; A61K 2800/85; C12R 2001/46

(57) ABSTRACT

Provided is a composition for skin containing, as an active component, an extract obtained by fermentation and aging with koji mold and two types of lactic acid bacteria. As a specific example, an extract obtained by adding water to koji mold (Aspergillus oryzae) to perform a primary fermentation for saccharification, then adding a lactic acid bacterium (Lactococcus cremoris SW01 stain) to perform a secondary fermentation, and then adding a nano-type lactic acid bacterium (Lactbacillus plantarum) is used as an active component.

### CROSS REFERENCE TO THE RELATED APPLICATIONS

This application is the national phase entry of International Application No. PCT/JP2023/038462, filed on Oct. 25, 2023, which is based upon and claims priority to Japanese Patent Application No. 2022-170889, filed on Oct. 25, 2022, the entire contents of which are incorporated herein by reference.

### TECHNICAL FIELD

The present invention relates to provision of a composition for skin, a skin improving agent, and a skin improving agent having a moisturizing function, which contain, as a main raw material, a fermented extract by microorganisms of koji mold and two types of lactic acid bacteria. The composition may be a composition for skin improvement containing, as a main component, Lactococcus cremoris itself or a fermented extract obtained by fermentation using Lactococcus cremoris.

### BACKGROUND

Skin is affected by an external environment such as infrared rays, ultraviolet rays, dryness, heat, and various pathogenic bacteria, and by an internal environment such as natural aging and diseases and viruses. As a result, it is known that phenomena such as wrinkles, macules, dryness, roughness, and rough skin occur. Many studies have been conducted to improve and prevent these skin issues and aging phenomena, and skin agents have been developed and commercialized.

In recent years, fermented extracts obtained by fermentation and aging by action of microorganisms are considered to be useful for maintaining and promoting health, and thus users and consumption of the fermented extracts have been increasing. Ever since, Food and drink products containing a fermented extract have been commercialized, and lately, basic skin care products which consist of fermented extracts as a main raw materials have also been commercialized, rather than food products that are ingested.

As a pharmacological action of fermented plant extracts, an aging-inhibiting action has been reported (Patent Literature 1). In addition, an inhibitory action on tyrosinase (melanin production inhibitory action), the production inhibitory action on AGE (an anti-glycation action), and the production inhibitory action on TNF-α and NO (an anti-inflammatory action) have also been reported (Patent Literature 2). A hyaluronic acid synthesis promoter containing a lactic acid bacterium fermentation product of pomegranate juice (Patent Literature 3) or a lactic acid bacterium fermentation product of a pulse extract (Patent Literature 4) as an active component has been reported. As described above, various beneficial effects on the skin are expected.

### CITATION LIST

### Patent Literatures

Patent Literature 1: JP 6013670 B1
Patent Literature 2: JP 2021-147361 A
Patent Literature 3: JP 2013-245170 A
Patent Literature 4: JP 2002-187838 A

### SUMMARY

### Technical Problem

So far, an extract obtained by stepwise fermentation of koji mold and two types of lactic acid bacteria has not been recognized. Further, little is known about effects on the skin of nano-type lactic acid bacteria (Lactbacillus plantarum) derived from sunkizuke pickles, which are eaten in the Kiso region of Nagano Prefecture.

The present inventors have intensively studied use and effects of fermented extracts and fermented compositions of various microorganisms. They have come up with the present invention based on a hypothesis that a beneficial fermented extract could be produced by individual and stepwise fermentation of a plurality of microorganism with different properties.

### Solution to Problem

Technical means used by the present invention to solve the technical problems are as follows.

Provided is a composition for skin containing, as an active component, an extract obtained by fermentation and aging with koji mold and two types of lactic acid bacteria. The lactic acid bacteria may be genus Lactococcus or Lactobacillus.

Provided is a composition for skin containing, as an active component, an extract obtained by adding water to Aspergillus oryzae to perform a primary fermentation for saccharification, then adding a lactic acid bacterium (Lactococcus cremoris H6 strain) to perform a secondary fermentation, and then adding a nano-type lactic acid bacterium (Lactbacillus plantarum).

As an active component, an extract obtained by sterilizing Aspergillus oryzae after the primary fermentation, sterilizing the lactic acid bacterium after the secondary fermentation, and then filtering to remove Aspergillus oryzae and the lactic acid bacterium may be used.

Aspergillus oryzae is often referred to as koji mold and rice malt.

A skin improving agent having a moisturizing function that contains the composition for skin as a main component may be used.

An elastase activity inhibitor containing the composition for skin as a main component may be used.

A collagen production promoter containing the composition for skin as a main component may be used.

A β-hexosaminidase activity inhibitor containing the composition for skin as a main component may be used. Further, a composition for skin improvement having a multifaceted effect, which is an elastase activity inhibitor, a collagen production promoter, and a β-hexosaminidase activity inhibitor, containing the composition for skin as a main component.

An external composition for skin containing the composition for skin as a main component may be used.

### Advantageous Effects of Invention

According to the present invention, it was possible to proceed with individual and stepwise fermentation processes of koji mold (Aspergillus oryzae) and lactic acid bacteria. In the fermentation processes of the present invention, a fermentation filtrate (a fermented extract, a fermented composition) was developed in which only the components produced from target koji mold and lactic acid bacteria were maximally extracted by microbial fermentation without using raw materials, additives, and additive agents other than the target koji mold and lactic acid bacteria.

This fermented extract/fermented composition suggests an effect of improving skin functions and preventing skin issues. Further, by adding the nano-type lactic acid bacterium (Lactbacillus plantarum) followed by these fermentation processes, the above preventive effect is predicted to be enhanced.

Hereinafter, the present invention will be further described by combining accompanying drawings and examples. The present invention is not limited to examples.

### BRIEF DESCRIPTION OF THE DRAWINGS

FIG. 1 is an explanatory diagram representing a process for producing a rice malt lactic acid bacterium fermentation liquid of the present invention.

FIG. 2 is a graph representing elastase activity in the rice malt lactic acid bacterium fermentation liquid (at various concentrations) of the present invention.

FIG. 3 is a graph representing collagen production in the rice malt lactic acid bacterium fermentation liquid of the present invention.

FIG. 4 is a graph representing inhibition of β-hexosaminidase activity in the rice malt lactic acid bacterium fermentation liquid of the present invention.

### DETAILED DESCRIPTION OF THE EMBODIMENTS

The present inventor focused on the microorganisms of koji mold and two types of lactic acid bacteria, and obtained a fermented extract (SW01, a test object of the present example) as described below.

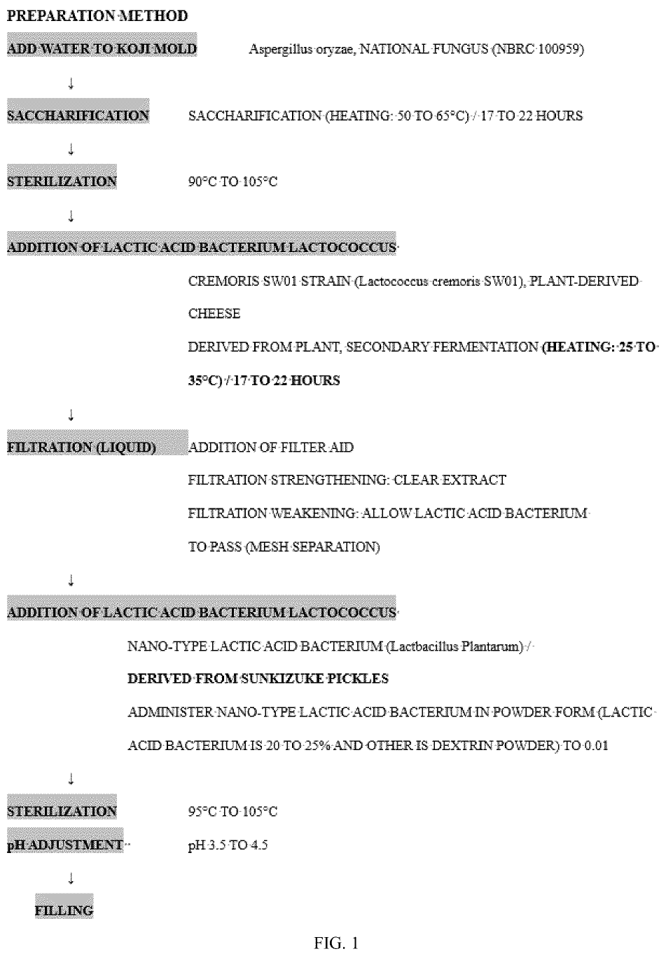

FIG. 1 is an explanatory diagram showing a fermentation process.

In the primary fermentation, about 1 to 20% of koji mold (including a culture medium) is added to and mixed with about 80 to 99% of water on a volume basis, and then fermentation is performed. The koji molds were Aspergillus oryzae and a national fungus (NBRC 100959). A fermentation temperature was 50° C. to 55° C., and a fermentation time was 17 hours to 22 hours.

When the fermentation time has elapsed, the temperature is raised from about 90° C. to 120° C., and the temperature is maintained for 10 minutes to 20 minutes to sterilize the koji mold at a high temperature, and this fermentation process is stopped.

The culture medium of the koji mold used in the present invention contains rice starch (powder) as a culture medium. In the primary fermentation, starch, which is a medium component (a rice starch component) outside a fungus body of the koji mold, is decomposed into monosaccharides such as glucose and saccharified using enzymes of the fungus.

When 1% rice malt was used, the Brix was 0.0 and the concentration of glucose was 0.03% before fermentation, but the Brix increased to 0.8 and the concentration of glucose increased to 0.38% after fermentation.

In this primary fermentation, it was shown that in the present case, the number of the fungus itself does not change because the koji mold only decomposes the rice starch, but a value of OD660 increases from 0.50 to 1.50. This correlates with an increase in the value of Brix.

The medium component (the rice starch component) outside the fungus body of the koji mold is saccharified by the koji mold and decomposed into monosaccharides such as glucose. Since this monosaccharide is used as a nutrient of lactic acid bacteria to be administered later, it is possible to produce a fermentation filtrate that does not use raw materials, additives, or additive agents that are not derived from koji mold and lactic acid bacteria.

Next, Lactococcus cremoris SW01 strain (NITE BP-03847) was added to the fermentation liquid in which this fermentation had been stopped to allow the fermentation by the lactic acid bacterium to proceed. A dose of the Lactococcus cremoris SW01 strain was 0.01 to 1% by weight of the total amount. The glucose that was saccharified earlier is used as a nutritional source for the lactic acid bacteria. The lactic acid bacterium is known to be derived from plant-derived cheese. The Lactococcus cremoris SW01 strain (SW01) was deposited at International Patent Organisms Depositary, National Institute of Technology and Evaluation on Mar. 3, 2023 (NITE BP-03847).

In the secondary fermentation using this lactic acid bacterium, the fermentation temperature was 25° C. to 35° C., and the fermentation time was 17 hours to 22 hours. After the fermentation time elapsed, the OD660 value became 1.00 to 2.00. The OD660 value showed that administered lactic acid has increased as well. The OD660 value depends on the number of bacterial cells, and an increase in the value means an increase in the number of bacterial cells.

After the fermentation time has elapsed, the temperature is raised from about 90 to 120° C., and the temperature is maintained for 10 minutes to 20 minutes to sterilize the lactic acid bacterium at a high temperature, then this fermentation process is stopped.

The pH of the fermentation liquid was 3.5 to 4.5, and insoluble matters such as rice residues were filtered in order to increase transparency of this fermented extract.

The level of filtration can be changed, and in order to enhance a filtration function, fossil mineral derived diatomaceous earth or volcanic rocks derived perlite can be used as a filter aid. To weaken a filtration function, mesh filtration is opted.

In this example, an OD value after filtration changed from 1.00 to 2.00 to 0.00 to 0.05. This filtration process removes insoluble matters such residues to obtain a fermentation filtrate (a fermented extract, a fermented composition).

As a final step, after filtration described above, a nano-type lactic acid bacterium (Lactbacillus plantarum) is administered to the fermentation liquid to obtain a concentration of 0.01 to 0.05%.

This nano-type lactic acid bacterium (Lactbacillus plantarum) is a sterilized lactic acid bacterium (the lactic acid bacterium is 20 to 25% and the rest is dextrin powder). Lactbacillus plantarum is known to be derived from Sunki pickles.

In general, lactic acid bacteria is disadvantageous as it aggregates when sterilized. To solve this disadvantage, nanosizing process (a high-pressure homogenization) deters aggregation to make it easier to handle as a raw material. After filtration, nano-type lactic acid bacteria were added to stabilize the components of the fermented extract (fermented composition).

Substances derived from nano-type lactic acid bacteria (cell walls, nucleic acids, proteins, sugars, etc.) were adopted because they may work effectively such as normalization of homeostatic functions by strengthening immunity on the epithelium (epidermis).

By adding the nano-type lactic acid bacterium (Lactbacillus plantarum), OD660 changes from 0.00-0.05 to 0.25-1.00 by adding nano-type lactic acid bacteria (Lactbacillus plantarum). Thereafter, this fermented extract (fermented composition) was maintained at 85 to 100° C. for 10 to 30 minutes to sterilize the fermented liquid. This process was adopted for preventing putrescence and contamination due to secondary contamination.

Next, a test was conducted to investigate the function or effect of the skin composition according to the present invention. First, an elastase activity inhibition test (anti-wrinkle test) was conducted.

Dermis that lies inside the epidermis is mainly made up of collagen, elastin, and hyaluronic acid. Produced by dermal fibroblasts, elastin plays an important role in maintaining skin flexibility and elasticity. Elastin is an “elastic fiber” that is flexible and stretchable (elastic), and has a property of returning to its original shape when an applied force is released. There is a coexistence relationship in which elastin bundles collagen.

Elastase, an elastin-degrading enzyme, is known to be induced by inflammation, aging and stimulation by ultraviolet rays, active oxygen, and stress, and when elastin is degraded by elastase, a cross-linked structure of collagen and elastin becomes brittle, elasticity of the skin is lost, and wrinkles are caused. Therefore, by inhibiting the activity of elastase, it can be expected to have an effect of restoring or maintaining dermal elasticity of the skin and aging.

Therefore, in this example, elastase activity was evaluated by measuring an amount of nitroaniline produced using elastase derived from human skin fibroblasts (NHDF-Ad), and using N-Succinyl-Ala-Ala-Ala-β-nitroanilide as a substrate. Based on the elastase activity, a skin dermis elasticity retention and aging function (an activity inhibition rate) of the test object of rice malt lactic acid bacterium fermentation liquid SW01 (hereinafter, SW01) was examined.

The elastase activity was set to 100% using, as a control, a reaction with only elastase derived from human skin fibroblasts (NHDF-Ad) and N-Succinyl-Ala-Ala-Ala-β-nitroanilide as a substrate. Reaction conditions in this experiment were 37° C. for 2 hours. Note that, phosphoramidon, a standard agent for elastase inhibition, was administered at 50 nM to show elastase inhibition, confirming that this experimental system functions effectively.

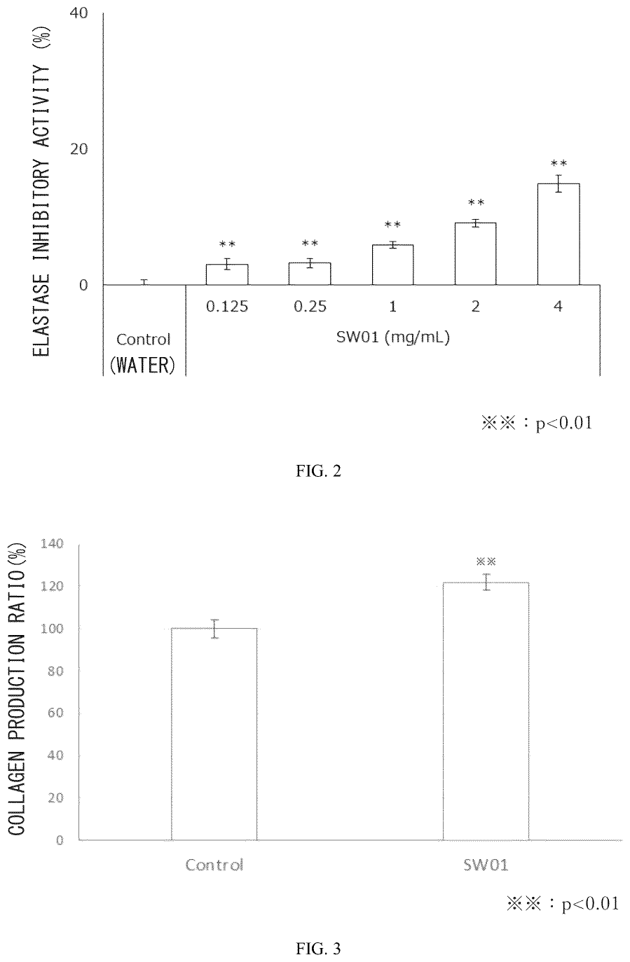

As a result, compared to the control, when SW01 as an evaluation sample was added at a concentration of 4 mg/mL, elastase inhibitory activity was observed to be 14.9% (p<0.01), and the inhibitory activity was further observed in a concentration-dependent manner (FIG. 2). Therefore, it was suggested that SW01 has the elastase inhibitory activity and has an effect of contributing to restoration of dermal elasticity of the skin or maintenance of an aging function.

Next, it was tested whether or not the rice malt lactic acid bacterium fermentation liquid SW01 promotes collagen production. SW01 (the sample) was added to human skin fibroblasts (NHDF-Ad), and the amount of collagen in a culture supernatant after 72 hours was measured using an ELISA method. As a result, compared to the control, the amount of collagen production per cell increased by 1.5 times in a test section to which 500 g/mL of SW01 was added (FIG. 3). It was shown that the rice malt lactic acid bacterium fermentation liquid SW01 promotes collagen production in a skin dermis layer.

From the above experimental contents, it was shown that the rice malt lactic acid bacterium fermentation liquid SW01 according to the present invention simultaneously exhibits two effects of inhibiting elastase and promoting collagen production. Therefore, it is confirmed that the effect of increasing the number of dermal fibroblasts and maintaining tension and the structure of the skin dermis is exhibited by applying the rice malt lactic acid bacterium fermentation liquid SW01 according to the present invention to the skin.

Next, it was tested whether or not the rice malt lactic acid bacterium fermentation liquid SW01 (the sample) contributes to anti-inflammatory and anti-allergy. IgE bound to the cell surface of adipocytes that is cross-linked by antigen is known to release granulocytes containing histamine and other substances to the outside of the cell, and canse an allergic reaction.

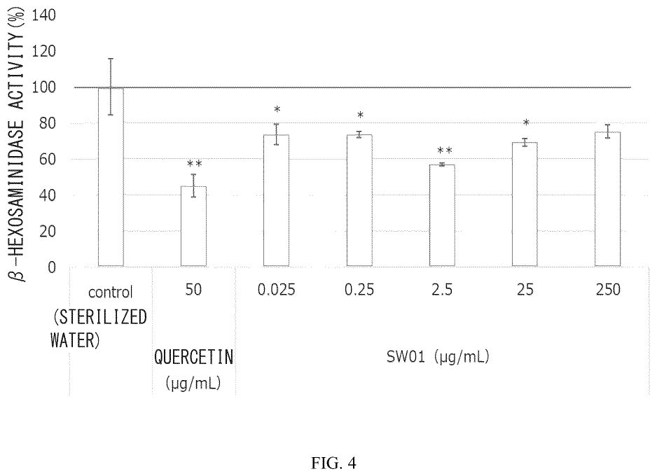

To determine an extent to which the test object inhibits degranulation, degranulation is assessed by measuring the activity of β-hexosaminidase, which is abundantly present in the granulocyte. Accordingly, the effect of the test object on allergic reaction is evaluated. It is known that rat basophilic leukemia cell RBL-2H3 releases the granulocyte containing histamine and the like to the outside of the cell when IgE bound to the cell surface is cross-linked with an antigen. This has been applied as an experimental system to exploratory research and the like as screening for substances having an anti-allergic action against type I allergy. In this example, the inhibition of degranulation is evaluated by stimulating RBL-2H3 cells with an antigen and measuring the activity of β-hexosaminidase (FIG. 4).

Assuming that the activity of β-hexosaminidase in a case where sterilized water was added dropwise as a control was 100%, the extent to which the activity of β-hexosaminidase was inhibited by adding the rice malt lactic acid bacterium fermentation liquid SW01 of the present invention as the test object was measured at various addition amounts. Further, 50 g/mL of quercetin was used as a positive control. In the positive control, the activity of β-hexosaminidase was about 40%, and this experimental system could be shown to be suitable. By adding 2.5 g/mL of the rice malt lactic acid bacterium fermentation liquid SW01, the activity of β-hexosaminidase was about 50%. It was shown that the rice malt lactic acid bacterium fermentation liquid SW01 inhibited the activity of β-hexosaminidase and exhibited an anti-allergic action and an anti-inflammatory action. Note that, the p value in FIG. 4 is p<0.01.

From the above, it was shown that the rice malt lactic acid bacterium fermentation liquid SW01 according to the present invention exhibits, as skin improving effects, various effects related to skin improvement such as inhibition of elastase activity, inhibition of β-hexosaminidase activity, and promotion of collagen production. The skin improvement includes improvement of presymptom by preventing minor disorder in the skin, such as itching and inflammation, in terms of health and hygiene.

### ACCESSION NUMBER

### National Institute of Technology and Evaluation

Room 122, 2-5-8 Kazusakamatari, Kisarazu-shi, Chiba, zip code 292-0818
Date of deposit: Mar. 3, 2023
Name of deposited microorganism: Lactococcus cremoris SW01
Accession number: BP-03847

| | | |
|---|---|---|
| 0-1 | This description of the deposited | JPO-PAS |
| 0-1-1 | microorganism or other biological material | i480 |
| | (PCT Rule 13bis) has been prepared by: | |
| 0-2 | International Application No. | |
| 0-3 | Applicant's or agent's docket number | C22164B |
| 1 | The indications made below relate to the | |
| | deposited microorganism or other biological | |
| | material referred to in the description | |
| 1-1 | Paragraph Number | [0048] |
| 1-3 | IDENTIFICATION OF DEPOSIT | |
| 1-3-1 | Name of depositary Institution | NITE Patent Microorganisms Depositary |
| 1-3-2 | Address of depositary institution | #122, 2-5-8, Kazusakamatari, Kisarazu-shi, |
| | | Chiba 292-0818 Japan |
| 1-3-3 | Date of deposit | 2023 Mar. 3 |
| 1-3-4 | Accession Number | NITE BP-03847 |
| 1-5 | DESIGNATED STATES FOR WHICH | All states: |
| | INDICATIONS ARE MADE | |

For receiving Office use only

| | | |
|---|---|---|
| 0-4 | This sheet was received with the | ✓ |
| | international application | |
| | (Yes/No) | |
| 0-4-1 | Authorized officer | Hirakawa Yuka |

For International Bureau use only

| | |
|---|---|
| 0-5 | This sheet was received by the |
| | International Bureau on: |
| 0-5-1 | Authorized officer |

What is claimed is:

1. A composition for skin comprising, as an active component, an extract obtained by a fermentation and an aging with koji mold and two types of lactic acid bacteria.

2. A composition for skin comprising, as an active component, an fermented extract obtained by a fermentation and an aging with koji mold and two types of lactic acid bacteria, wherein the two types of lactic acid bacteria belong to genus Lactococcus.

3. The composition for the skin according to claim 1 comprising, as the active component, the extract obtained by adding water to Aspergillus oryzae to perform a primary fermentation for a saccharification, then adding a lactic acid bacterium to perform a secondary fermentation, and then adding a nano-type lactic acid bacterium, wherein the lactic acid bacterium is Lactococcus cremoris and the nano-type lactic acid bacterium is Lactbacillus plantarumm.

4. The composition for the skin according to claim 3 comprising, as the active component, the extract obtained by sterilizing the Aspergillus oryzae after the primary fermentation, sterilizing the lactic acid bacterium after the secondary fermentation, and then filtering to remove the Aspergillus oryzae and the lactic acid bacterium.

5. A skin improving agent having a moisturizing function, comprising the composition for the skin according to claim 1 as a main component.

6. An elastase activity inhibitor, comprising the composition for the skin according to claim 1 as a main component.

7. A collagen production promoter, comprising the composition for the skin according to claim 1 as a main component.

8. A β-hexosaminidase activity inhibitor, comprising the composition for the skin according to claim 1 as a main component.

9. An external composition for skin, comprising the composition for the skin according to claim 1 as a main component.

10. A composition for a skin improvement comprising, as a main component, a fermented extract obtained by a fermentation using Lactococcus cremoris.

11. The composition for the skin according to claim 2 comprising, as the active component, the fermented extract obtained by adding water to Aspergillus oryzae to perform a primary fermentation for a saccharification, then adding a lactic acid bacterium to perform a secondary fermentation, and then adding a nano-type lactic acid bacterium, wherein the lactic acid bacterium is Lactococcus cremoris and the nano-type lactic acid bacterium is Lactbacillus plantarum.

12. The composition for the skin according to claim 11 comprising, as the active component, the fermented extract obtained by sterilizing the Aspergillus oryzae after the primary fermentation, sterilizing the lactic acid bacterium after the secondary fermentation, and then filtering to remove the Aspergillus oryzae and the lactic acid bacterium.
